(19) United States
(12) Patent Application Publication
(10) Pub. No.: US 2025/0345020 A1
(43) Pub. Date: Nov. 13, 2025

(54) NUCLEAR MEDICINE DIAGNOSTIC APPARATUS

(71) Applicant: CANON MEDICAL SYSTEMS CORPORATION, Tochigi (JP)

(72) Inventor: Masaki MIYAHARA, Otawara (JP)

(73) Assignee: CANON MEDICAL SYSTEMS CORPORATION, Tochigi (JP)

(21) Appl. No.: 19/196,853

(22) Filed: May 2, 2025

(30) Foreign Application Priority Data
May 7, 2024 (JP) 2024-075297

(51) Int. Cl. A61B 6/00; A61B 6/03

(52) U.S. Cl. CPC A61B 6/541; A61B 6/032; A61B 6/037; A61B 6/5264

(57) ABSTRACT

A nuclear medicine diagnostic apparatus includes processing circuitry configured to analyze respiration motion based on list mode data obtained through nuclear medicine scan on a subject having respiration motion and heartbeat motion, generate first images from respective division data sets obtained by dividing the list mode data for respective respiration phases as phases of the respiration motion, analyze the heartbeat motion for the respective respiration phases based on the list mode data, and generate second images from respective division data sets obtained by dividing phase data, obtained by dividing the list mode data for the respective respiration phases, for respective cardiac pulsation phases as phases of the heartbeat motion.

### CROSS-REFERENCE TO RELATED APPLICATIONS

This application is based upon and claims the benefit of priority from Japanese Patent Application No. 2024-075297, filed May 7, 2024, the entire contents of which are incorporated herein by reference.

### FIELD

Exemplary embodiments disclosed in the present specification and the drawings relate to a nuclear medicine diagnostic apparatus.

### BACKGROUND

Image gathering by a nuclear medicine diagnostic apparatus may be affected by the respiration motion and heartbeat motion. Synchronous reconstruction techniques such as a respiration synchronous reconstruction and a cardiac pulsation synchronous reconstruction are known to suppress the influences of such periodic movements to improve the image quality. For example, by measuring the respiration and cardiac pulsation with external devices attached to a subject, the reconstruction techniques associate data collected by a nuclear medicine diagnostic apparatus with respiration phases and cardiac pulsation phases. Then, by extracting data of specific phases and using the data for reconstruction processing, the techniques enable obtaining nuclear medicine images with restricted influences of the respiration motion and heartbeat motion. However, attaching external devices to a subject causes a burden on both the subject and the operator such as a technician.

### BRIEF DESCRIPTION OF THE DRAWINGS

FIG. 1 illustrates an example configuration of a nuclear medicine diagnostic apparatus according to an exemplary embodiment.

FIG. 2 is a flowchart illustrating a series of processing of processing circuitry according to a first exemplary embodiment.

FIG. 3A illustrates an example of respiration motion analysis according to the first exemplary embodiment.

FIG. 3B illustrates another example of respiration motion analysis according to the first exemplary embodiment.

FIG. 4A illustrates still another example of respiration motion analysis according to the first exemplary embodiment.

FIG. 4B illustrates still another example of respiration motion analysis according to the first exemplary embodiment.

FIG. 5 illustrates images for different respiration phases according to the first exemplary embodiment.

FIG. 6 illustrates motion vectors according to the first exemplary embodiment.

FIG. 7 illustrates phase data according to the first exemplary embodiment.

FIG. 8 illustrates an example of processing for estimating a cardiac pulsation waveform according to the first exemplary embodiment.

FIG. 9 illustrates processing for generating single-phase images according to the first exemplary embodiment.

FIG. 10 illustrates processing for generating single-phase images according to a second exemplary embodiment.

FIG. 11 illustrates an example configuration of a medical information processing system according to a third exemplary embodiment.

### DETAILED DESCRIPTION

A nuclear medicine diagnostic apparatus according to an embodiment includes processing circuitry configured to analyze respiration motion based on list mode data obtained through nuclear medicine scan on a subject having respiration motion and heartbeat motion, generate first images from respective division data sets obtained by dividing the list mode data for respective respiration phases as phases of the respiration motion, analyze the heartbeat motion for the respective respiration phases based on the list mode data, and generate second images from respective division data sets obtained by dividing phase data, obtained by dividing the list mode data for the respective respiration phases, for respective cardiac pulsation phases as phases of the heartbeat motion.

Various Embodiments will be described hereinafter with reference to the accompanying drawings.

Exemplary embodiments of a nuclear medicine diagnostic apparatus will be described below with reference to the accompanying drawings.

### First Exemplary Embodiment

A nuclear medicine diagnostic apparatus 10 will be described below. The nuclear medicine diagnostic apparatus 10 is a medical image diagnostic apparatus (modality) capable of executing nuclear medicine scan. Nuclear medicine scan refers to scan performed by giving a medical agent labelled with a radionuclide to a subject. Examples of typical scans include the Positron Emission Computed Tomography (PET) scan and the Single Photon Emission Computed Tomography (SPECT) scan.

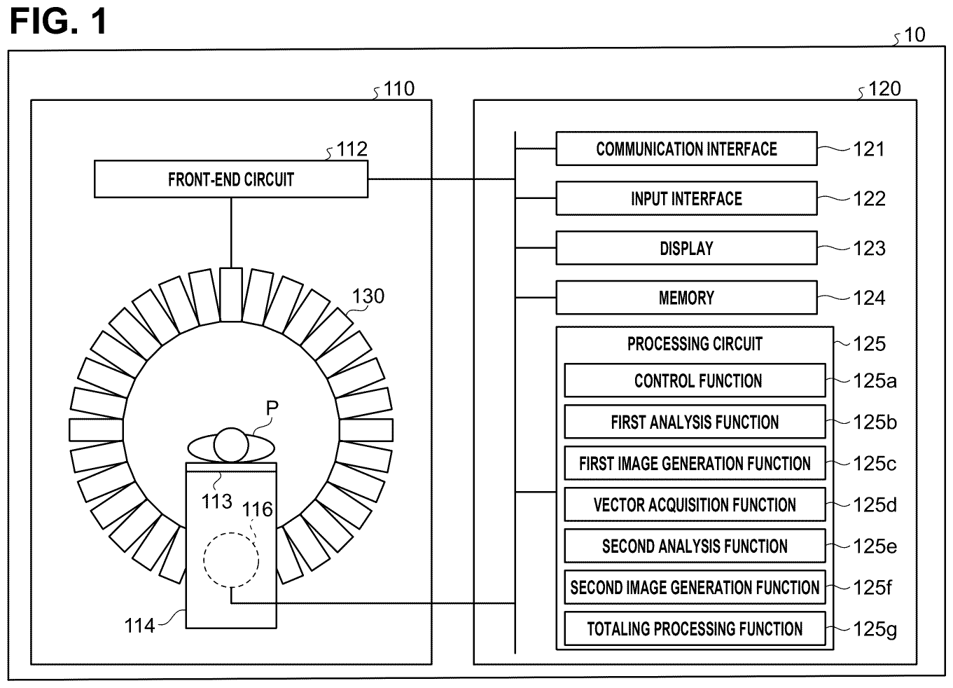

The present exemplary embodiment will be described below with reference to FIG. 1, centering on a case where the nuclear medicine diagnostic apparatus 10 is a PET apparatus. FIG. 1 illustrates an example configuration of the nuclear medicine diagnostic apparatus 10 according to a first exemplary embodiment. The nuclear medicine diagnostic apparatus 10 illustrated in FIG. 1 includes a base apparatus 110 and a console apparatus 120. The base apparatus 110 includes a detector 130, front-end circuitry 112, a couchtop 113, a couch 114, and a couch drive unit 116.

The detector 130 detects a radiation. For example, the detector 130 detects a radiation by detecting scintillation light (fluorescence) which is released again when a material that turned into the excited state through an interaction between a gamma ray and an illuminant makes a state transition back to the ground state. A gamma ray is generated when positive electrons released from accumulated medical agents given to the subject P cause pair annihilation with electrons in the surrounding tissue. According to the exemplary embodiment, the detector 130 can also detect a Cherenkov light. The detector 130 detects radiological energy information of a gamma ray generated when positive electrons released from the accumulated medical agents given to the subject P cause pair annihilation with electrons in the surrounding tissue. A plurality of the detectors 130 is disposed to surround the subject P in a ring form, for example, to form a plurality of detector blocks.

Typically, the detector 130 includes a scintillator crystal and a light detection surface formed of light detection elements. Examples of materials for the scintillator crystal include materials suitable for producing a Cherenkov light, such as Bismuth Germanium Oxide (BGO), lead glass (SiO2+PbO), lead fluoride (PbF2), PWO (PbWO4), and other lead compounds. Other examples include Lutetium Yttrium Oxyorthosilicate (LYSO), Lutetium Oxyorthosilicate (LSO), Lutetium Gadolinium Oxyorthosilicate (LGSO), and scintillator crystals such as BGO. Each of the light detection elements forming the light detection surface includes, for example, a plurality of pixels each of which is formed of, for example, Single Photon Avalanche Diodes (SPADs). The configuration of the detector 130 is not limited to the above-mentioned example. The light detection elements may be, for example, Silicon photoelectron multipliers (SiPMs) or photoelectron multipliers. The scintillator crystal may be a monolithic crystal, and the light detection surface formed of light detection elements may be disposed, for example, on the six facets of the scintillator crystal.

The base apparatus 110 generates counting (number of counts) information based on the output signal of the detector 130 via the front-end circuitry 112, and stores the generated counting information in a memory 124 of the console apparatus 120. The detector 130 may be divided into a plurality of blocks each of which includes the front-end circuitry 112.

The front-end circuitry 112 converts the output signal from the detector 130 into digital data, and generates the counting information. For example, this counting information includes the detecting position, energy value, and detection time of an annihilation gamma ray. For example, the front-end circuitry 112 identifies a plurality of light detection elements that convert scintillation light into an electrical signal at the same timing. The front-end circuitry 112 identifies the scintillator number (P) indicating the scintillator position on which an annihilation gamma ray is incident. A method for identifying the scintillator position on which an annihilation gamma ray is incident may be one that employs center-of-gravity calculation based on the position of each light detection element and the intensity of the electrical signal. If the scintillator corresponds to the size of each light detection element, for example, the method assumes the scintillator corresponding to the light detection element providing the maximum output, as the scintillator position on which an annihilation gamma ray is incident, and finally identifies the scintillator position in consideration of the scattering between scintillators.

The front-end circuitry 112 integrates the intensity of the electrical signal output from each light detection element or measures the time during which the electrical signal intensity exceeds a threshold value (Time over Threshold), identifies the energy value (E) of the annihilation gamma ray incident on the detector 130. The front-end circuitry 112 also identifies the detection time (T) when scintillation light by an annihilation gamma ray is detected by the detector 130. The detection time (T) may be the absolute time or elapsed time since the imaging start timing. Thus, the front-end circuitry 112 generates counting information including the scintillator number (P), energy value (E), and detection time (T).

For example, the front-end circuitry 112 is implemented by such circuitry as a Central Processing Unit (CPU), Graphical Processing Unit (GPU), Application Specific Integrated Circuit (ASIC), and Programmable Logic Device (PLD) (such as a Simple Programmable Logic Device (SPLD), Complex Programmable Logic Device (CPLD), and Field Programmable Gate Array (FPGA)).

The couchtop 113, a bed on which the subject P is laid, is disposed on the couch 114. The couch drive unit 116 moves the couchtop 113 under the control of a control function 125a of the processing circuitry 125. For example, the couch drive unit 116 moves the couchtop 113 to move the subject P into the imaging opening of the base apparatus 110.

The console apparatus 120 receives operations on the nuclear medicine diagnostic apparatus 10 by the operator, and controls execution of nuclear medicine scan, and reconstructs nuclear medicine images based on the collected list mode data. If the nuclear medicine diagnostic apparatus 10 is a PET apparatus, the console apparatus 120 controls execution of PET scan to reconstruct PET images based on the list mode data. As illustrated in FIG. 1, the console apparatus 120 includes a communication interface 121, an input interface 122, a display 123, the memory 124, and the processing circuitry 125.

The communication interface 121 controls transmission and communication of various types of data transmitted and received between other apparatuses and systems connected with the console apparatus 120 via a network. More specifically, the communication interface 121 is connected with the processing circuitry 125 and outputs data received from other apparatuses and systems to the processing circuitry 125 or outputs data output from the processing circuitry 125 to other apparatuses and systems. For example, the communication interface 121 is implemented by a network card, a network adapter, or a Network Interface Controller (NIC).

The input interface 122 receives various input operations from the operator, converts the received input operations into electrical signals, and outputs the signals to the processing circuitry 125. For example, the input interface 122 is implemented by a mouse, keyboard, track ball, switches, buttons, joystick, touch pad, which is used to perform input operations by touching the operation panel, touch screen with an integrated display screen and a touch pad, noncontact input circuitry using an optical sensor, and audio input circuitry. The input interface 122 may be formed of a tablet terminal capable of wirelessly communicating with the main body of the console apparatus 120. The input interface 122 may also be circuitry for receiving input operations from the operator through a motion capture. For example, the input interface 122 can receive the body movement or line of sight of the operator by processing the signals obtained via a tracker and images collected for the operator. The input interface 122 is not limited to an interface having a mouse, a keyboard, and other physical operation members. Examples of the input interface 122 also includes electrical signal processing circuitry for receiving an electrical signal corresponding to an input operation from an external input device separately provided from the console apparatus 120, and outputting the electrical signal to the processing circuitry 125.

The display 123 displays various information such as nuclear medicine images, respiration waveforms, cardiac pulsation waveforms, and other various medical information collected from the subject P. The display 123 also displays Graphical User Interfaces (GUIs) used to receive various instructions and settings from the operator via the input interface 122. For example, the display 123 is a Liquid Crystal Display (LCD) or a Cathode Ray Tube (CRT) display. The display 123 may also be a desktop display or a tablet terminal capable of wirelessly communicating with the main body of the console apparatus 120.

The memory 124 is implemented by a semiconductor memory element (such as a Random Access Memory (RAM) and a flash memory), a hard disk, and an optical disk. For example, the memory 124 stores various medical information and programs required to implement the functions of the circuitry included in the console apparatus 120. The memory 124 may be implemented by a server group (cloud) connected with the console apparatus 120 via a network NW.

The processing circuitry 125 functions as a control function 125a, a first analysis function 125b, a first image generation function 125c, a vector acquisition function 125d, a second analysis function 125e, a second image generation function 125f, and a totaling processing function 125g to control the overall operations of the console apparatus 120. For example, the processing circuitry 125 reads the program corresponding to the control function 125a from the memory 124 and executes the program to function as the control function 125a.

For example, the control function 125a controls the operations of various components included in the base apparatus 110 to execute nuclear medicine scan on the subject P and acquire list mode data indicating the result of radiological detection. The control function 125a also controls display of the display 123 and controls data transmission and reception via the network NW. For example, the control function 125a transmits the list mode data collected through nuclear medicine scan and nuclear medicine images reconstructed based on the list mode data to the server of a Picture Archiving and Communication System (PACS) for registration.

Likewise, the processing circuitry 125 functions as the first analysis function 125b, the first image generation function 125c, the vector acquisition function 125d, the second analysis function 125e, the second image generation function 125f, and the totaling processing function 125g. The first analysis function 125b is an example of a first analysis unit. The first image generation function 125c is an example of a first image generation unit. The vector acquisition function 125d is an example of a vector acquisition unit. The second analysis function 125e is an example of a second analysis unit. The second image generation function 125f is an example of a second image generation unit. The totaling processing function 125g is an example of a totaling processing unit. Processing by the first analysis function 125b, the first image generation function 125c, the vector acquisition function 125d, the second analysis function 125e, the second image generation function 125f, and the totaling processing function 125g will be described in detail below.

In the nuclear medicine diagnostic apparatus 10 illustrated in FIG. 1, the processing functions are stored in the memory 124 in a form of computer-executable programs. The processing circuitry 125 is a processor that reads a program from the memory 124 and executes the program to implement the function corresponding to each program. In other words, the processing circuitry 125 having read a program is provided with the function corresponding to the read program.

FIG. 1 illustrates that the single processing circuitry 125 implements the control function 125a, the first analysis function 125b, the first image generation function 125c, the vector acquisition function 125d, the second analysis function 125e, the second image generation function 125f, and the totaling processing function 125g. However, the processing circuitry 125 may be configured by combining a plurality of independent processors each of which executes a program to implement the corresponding function. Each of the processing functions of the processing circuitry 125 may be suitably implemented by a single or a plurality of pieces of processing circuitry in a distributed or integrated way.

The processing circuitry 125 may implement the functions by using the processors of external devices connected via the network NW. For example, the processing circuitry 125 implements each function illustrated in FIG. 1 by reading a program corresponding to each function from the memory 124 and executing the program, and using, as calculation resources, a server group (cloud) connected with the nuclear medicine diagnostic apparatus 10 via the network NW.

An example configuration of the nuclear medicine diagnostic apparatus 10 has been described above. Depending on the imaging range, the list mode data collected through nuclear medicine scan may be affected by the movements of the subject P, such as the respiration motion and heartbeat motion. When performing reconstruction processing without taking the influences of such movements into consideration, a region including any movement in nuclear medicine images to be reconstructed may become defocused, possibly disturbing diagnoses.

A respiration synchronous reconstruction and a cardiac pulsation synchronous reconstruction are known as techniques for reducing the influences of the respiration motion and heartbeat motion to improve the image quality of nuclear medicine images. More specifically, when performing the synchronous reconstruction, external devices such as a respiration synchronous monitor and an electrocardiogram synchronous monitor are attached to the subject P before starting nuclear medicine scan. This enables measuring the respiration and cardiac pulsation of the subject P by using external devices while collecting the list mode data through nuclear medicine scan, thus associating the list mode data with the respiration and cardiac pulsation phases.

Then, by extracting data of specific phases from the list mode data and using the data for the reconstruction processing, nuclear medicine images with the restricted influences of the respiration and cardiac pulsation can be obtained. For example, the movement of the subject P by respiration decreases in the phase at the end of exhalation. Therefore, by extracting data corresponding to the phase at the end of exhalation out of the list mode data and using the data for the reconstruction processing, respiration synchronous images with the restricted influence of the respiration motion can be obtained.

However, using external devices for the synchronous reconstruction causes a burden on both the operator and the subject P. For example, the process of attaching external devices to the subject P and the process of detaching them from the subject P will degrade the work flow, prolonging the inspection time. When attaching the electrodes of external devices to the skin of the subject P, the electrodes may adversely affect the skin. In addition, there arises costs for purchasing and maintaining external devices.

For this reason, a technique for performing the synchronous reconstruction by analyzing the list mode data without using external devices is currently being studied. The technique is also referred to as a device-less synchronous reconstruction or a data-driven synchronous reconstruction.

However, since the list mode data may be compositely affected by a plurality of periodic movements having different frequencies, such as the respiration motion and heartbeat motion, it is difficult to accurately execute the device-less synchronous reconstruction. For example, when performing a device-less respiration synchronous reconstruction, a respiration waveform is estimated based on the list mode data. In this case, the accuracy of the respiration waveform estimation may be degraded by the influence of the heartbeat motion. Since the respiration motion is generally larger in movement than the heartbeat motion, the respiration motion is predominantly detected in the analysis of the list mode data. This means that the cardiac pulsation synchronous reconstruction is difficult particularly when performing the device-less synchronous reconstruction.

On the other hand, the processing of the processing circuitry 125 (described in detail below) enables the nuclear medicine diagnostic apparatus 10 to perform the synchronous reconstruction for periodic movements of the subject P without using external devices.

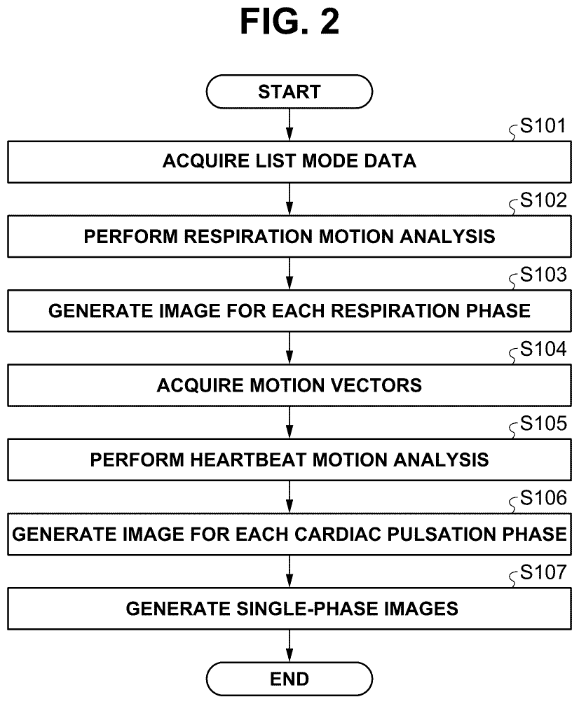

FIG. 2 illustrates a series of processing by the processing circuitry 125. FIG. 2 is a flowchart illustrating a series of processing by the processing circuitry 125 according to the first exemplary embodiment.

In step S101, the control function 125a acquires the list mode data. The list mode data is counting information collected from the subject P through nuclear medicine scan, and is also referred to as raw data. The format of the list mode data is not limited to a specific format. The list mode data includes at least information about the detection time.

For example, the control function 125a acquires the list mode data by controlling the operations of various components included in the base apparatus 110 and executing nuclear medicine scan on the subject P. Alternatively, the control function 125a may acquire the list mode data collected in advance by the nuclear medicine diagnostic apparatus 10 or other nuclear medicine diagnostic apparatuses. For example, the control function 125a may acquire the list mode data registered on the PACS server via the network NW.

In step S102, the first analysis function 125b analyzes the respiration motion based on the list mode data. More specifically, the first analysis function 125b performs the respiration motion analysis based on the list mode data obtained through nuclear medicine scan on the subject P having the respiration motion and heartbeat motion.

Referring to the example illustrated in FIG. 2, in step S105 after step S102 (respiration motion analysis), the first analysis function 125b executes heartbeat motion analysis (described below). When analyzing two different periodic movements, a first periodic movement to be analyzed first needs to be longer in period than a second periodic movement. More specifically, when analyzing two different movements (respiration motion and heartbeat motion), the respiration motion is a first analysis target and the heartbeat motion is a second analysis target.

For example, the first analysis function 125b extracts a part of the list mode data in the time direction as partial data, and estimates a respiration waveform based on reconstruction images reconstructed based on the extracted partial data. More specifically, the first analysis function 125b estimates a respiration waveform based on a plurality of time-division reconstruction images. For example, the first analysis function 125b can estimate a respiration waveform by analyzing the correlation between reconstruction images through principal component analysis. The estimated respiration waveform may be displayed on the display 123 under the control of the control function 125a.

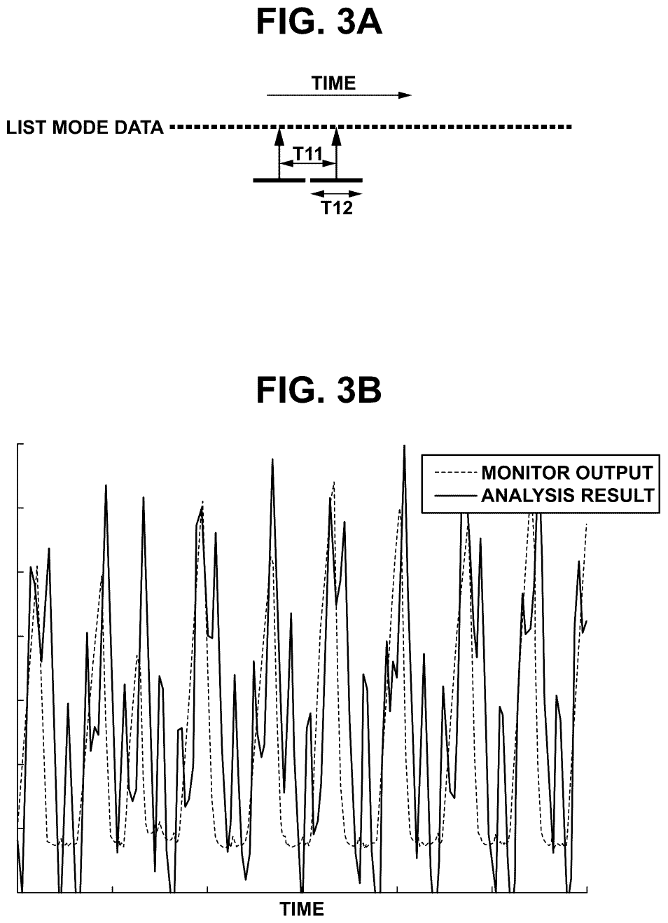

An example of respiration motion analysis will be described below with reference to FIGS. 3A and 3B. Referring to FIG. 3A, a time interval T11 is set as a time interval for respiration motion analysis, and a time duration T12 is set as a time duration for respiration motion analysis. The time duration refers to the size of partial data in the time direction to be extracted from the list mode data. With decreasing time duration, the analysis time resolution improves and the statistical noise influence increases. The time interval indicates the frequency of extracting partial data from the list mode data, i.e., a sampling interval in the time direction. The first analysis function 125b extracts partial data having the time duration T12 from the list mode data at time intervals T11, and estimates a respiration waveform based on reconstruction images reconstructed based on the extracted partial data.

Referring to FIGS. 3A and 3B, the settings of the time interval T11 and the time duration T12 are reduced to a degree that the heartbeat motion having a higher frequency movement than the respiration motion can be detected. For example, the time intervals T11 and the time duration T12 are set to about “0.2 seconds”. The solid line chart in FIG. 3B indicates the result of the respiration waveform estimation when analysis is performed with the above-described settings. The broken line chart in FIG. 3B indicates the output from the respiration synchronous monitor. The output from the respiration synchronous monitor illustrated in FIG. 3B will be described below on the premise that the output is obtained by approximating the actual respiration waveform to a degree that the respiration synchronous reconstruction having sufficient accuracy can be implemented. The analysis result illustrated in FIG. 3B includes a number of peaks produced by the influence of the cardiac pulsation, which do not exist in the actual respiration waveform. Accordingly, the accuracy of the result of the respiration waveform estimation is not high.

To prevent the influence of the cardiac pulsation in the respiration waveform estimation, the settings of the time interval T11 and the time duration T12 may possibly be prolonged. For example, the time interval T11 and the time duration T12 may be set to about “1.0 seconds”. If the time duration T12 prolongs to allow each of partial data sets to include about one period or more of the cardiac pulsation, the influence of the cardiac pulsation is smoothed and hence a smooth chart can be obtained as a result of the respiration waveform estimation. However, the estimation result is a rough estimation result obtained at coarse sampling intervals. Accordingly, the accuracy of the result of the respiration waveform estimation is not high.

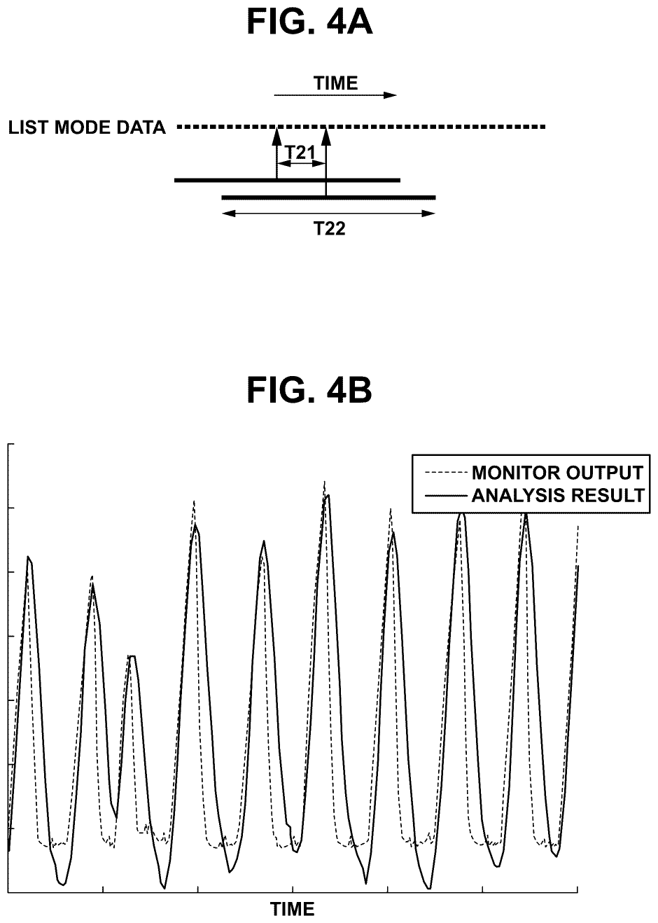

Therefore, as illustrated in FIG. 4A, the first analysis function 125b may set a time interval T21 for respiration motion analysis and a time duration T22 for respiration motion analysis which is longer than the time interval T21. For example, the first analysis function 125b sets the time interval T21 to about “0.2 seconds”, and sets the time duration T22 to about “1.0 second”. As illustrated by the solid line chart in FIG. 4B, this technique enables smoothing the influence of the cardiac pulsation to obtain a smooth chart, and accurately estimating a respiration waveform at sufficient sampling intervals.

Referring to FIG. 4B, the solid line chart illustrating the respiration waveform estimated with the device-less synchronous reconstruction approximately coincides with the broken line chart illustrating the output from the respiration synchronous monitor. This means that estimating a respiration waveform with the setting illustrated in FIG. 4A enables executing the respiration synchronous reconstruction with almost the same accuracy as that in the case of using the respiration synchronous monitor.

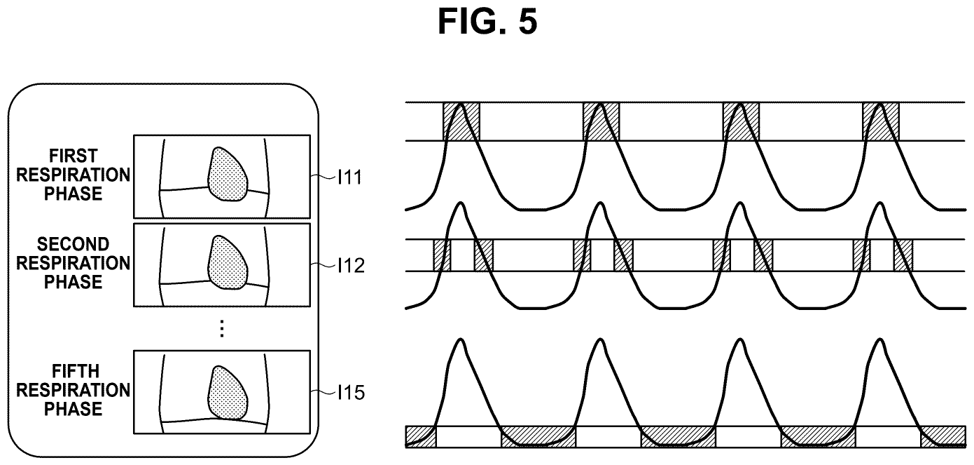

In step S103, the first image generation function 125c generates an image for each respiration phase, i.e., the first image generation function 125c performs the respiration synchronous reconstruction. The processing in step S103 will be described below with reference to FIG. 5. FIG. 5 illustrates an image for each respiration phase according to the first exemplary embodiment. FIG. 5 illustrates a case where five different phases are included in the respiration period. More specifically, the number of phases included in the respiration period and the definition of each phase can be suitably changed, automatically adjusted by the apparatus, or manually changed by the operator.

The charts on the right-hand side of FIG. 5 illustrate the respiration waveforms estimated in step S102. The rectangles superposed on the charts indicate time ranges corresponding to respective respiration phases. For example, the four rectangles superposed on the top chart out of the three charts in FIG. 5 indicate time ranges corresponding to the first respiration phase. The first image generation function 125c divides the list mode data included in the time ranges indicated by these four rectangles and reconstructs an image I11 of the first respiration phase from the division data.

Likewise, the first image generation function 125c generates images of different phases including an image I12 of a second respiration phase and an image I15 of a fifth respiration phase. Although not illustrated in FIG. 5, the first image generation function 125c also generates an image of a third respiration phase and an image of a fourth respiration phase.

According to the present exemplary embodiment, the images I11, I12, and I15, the image of the third respiration phase (not illustrated), and the image of the fourth respiration phase (not illustrated) are examples of first images. More specifically, the first image generation function 125c generates a first image based on each of division data sets obtained by dividing the list mode data for respective respiration phases. Each of the first images such as the image I11 is, for example, an image obtained by smoothing about one period of the cardiac pulsation.

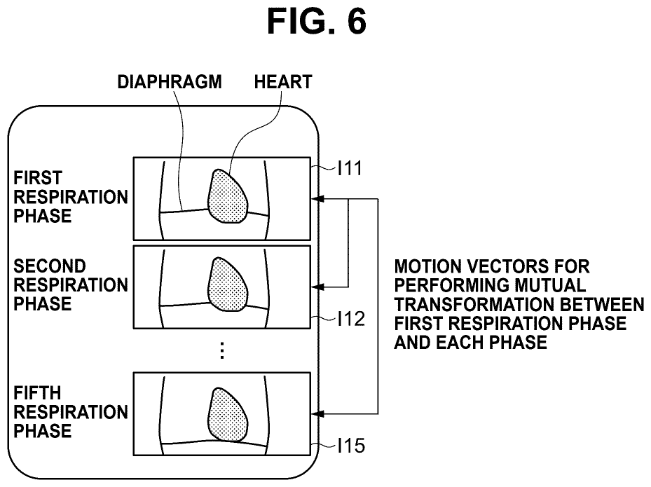

In step S104, the vector acquisition function 125d acquires motion vectors indicating variations between respiration phases based on the first images generated in step S103. The motion vectors indicating variations between respiration phases are also referred to as first motion vectors. The processing in step S104 will be described below with reference to FIG. 6. FIG. 6 illustrates an example of acquiring motion vectors for performing mutual transformation between the first respiration phase and other phases by using the first respiration phase as a reference phase.

For example, when comparing the image I12 of the second respiration phase with the image I11 of the first respiration phase, feature point positions such as the cardiac position relative to the diaphragm are different. The vector acquisition function 125d acquires a motion vector to offset such differences. For example, the vector acquisition function 125d performs registration processing between images to acquire the inverse transformation of the registration processing as a motion vector. The vector acquisition function 125d may perform the registration processing or motion vector acquisition processing itself by using a machine learning technique (such as neural networks).

For example, in a case where a certain feature point has position coordinates (x1, y1, z1) in the image I11 and position coordinates (x2, y2, z2) in the image I12, position coordinates (x1-x2, y1-y2, z1-z2) can be obtained as a motion vector of the feature point. The position coordinates may be defined by providing an origin in the image I11 or by providing an origin on an imaging space such as the couchtop 113 and the floor. A feature point refers to, for example, an anatomical feature point on the heart or diaphragm.

A motion vector may be acquired for each position (e.g., each pixel) on an image or acquired for each feature point. FIG. 6 illustrates an example of obtaining motion vectors for performing mutual transformation between a reference phase and other phases. However, the vector acquisition function 125d may acquire a motion vector for performing mutual transformation between phases. For example, the vector acquisition function 125d may acquire motion vectors for performing mutual transformation between the first respiration phase and other phases, motion vectors for performing mutual transformation between the second respiration phase and other phases, motion vectors for performing mutual transformation between the third respiration phase and other phases, motion vectors for performing mutual transformation between the fourth respiration phase and other phases, and motion vectors for performing mutual transformation between the fifth respiration phase and other phases.

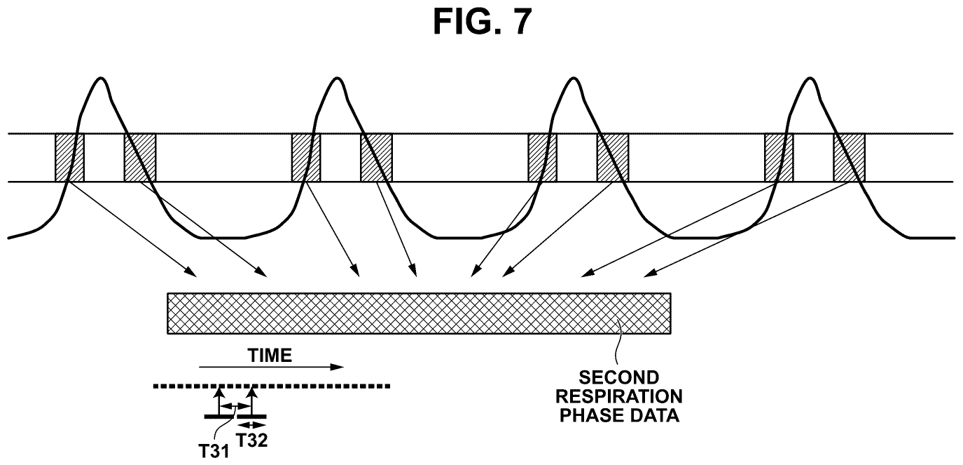

In step S105, the second analysis function 125e performs heartbeat motion analysis for each respiration phase based on the list mode data. More specifically, the second analysis function 125e firstly divides the list mode data for respective respiration phases. For example, as illustrated in FIG. 7, the second analysis function 125e divides the list mode data included in the time ranges of the second respiration phase to acquire second respiration phase data. The second respiration phase data is an example of phase data.

The second analysis function 125e further extracts a part of the phase data in the time direction as partial data, and estimates a cardiac pulsation waveform based on a reconstruction image reconstructed based on the extracted partial data. The phase data refers to respiration synchronous data in which the influence of the respiration motion has been eliminated or prevented. Therefore, estimating a cardiac pulsation waveform based on the phase data enables eliminating or preventing the influence of the respiration motion in heartbeat motion analysis, thus making accurate estimation of a cardiac pulsation waveform possible. The estimated cardiac pulsation waveform may be displayed on the display 123 under the control of the control function 125a.

For example, as illustrated in FIG. 7, the second analysis function 125e extracts partial data having a time duration T32 from the second respiration phase data at time intervals T31. The settings of the time interval T31 and the time duration T32 are reduced to a degree that the heartbeat motion can be detected. For example, the time interval T31 and the time duration T32 are set to about “0.2 seconds”.

Although FIG. 7 illustrates the time interval T31 and the time duration T32 having the same length, the present exemplary embodiment is not limited thereto. For example, the time interval T31 may be set to about “0.1 seconds” and the time duration T32 may be set to about “0.2 seconds”. This enables accurately estimating a cardiac pulsation waveform at shorter sampling intervals while preventing the influence of statistical noise from excessively increasing.

FIG. 7 illustrates an example of dividing the data included in the time ranges of the second respiration phase to acquire the second respiration phase data. However, the second analysis function 125e can also acquire phase data of other respiration phases in a similar way. For example, by dividing the data included in the time ranges of the first respiration phase, the second analysis function 125e can acquire the first respiration phase data from the list mode data. Further, the second analysis function 125e extracts a part of the first respiration phase data in the time direction as partial data, and estimates a cardiac pulsation waveform based on a reconstruction image reconstructed based on the extracted partial data. More specifically, the second analysis function 125e can estimate a cardiac pulsation waveform for each respiration phase.

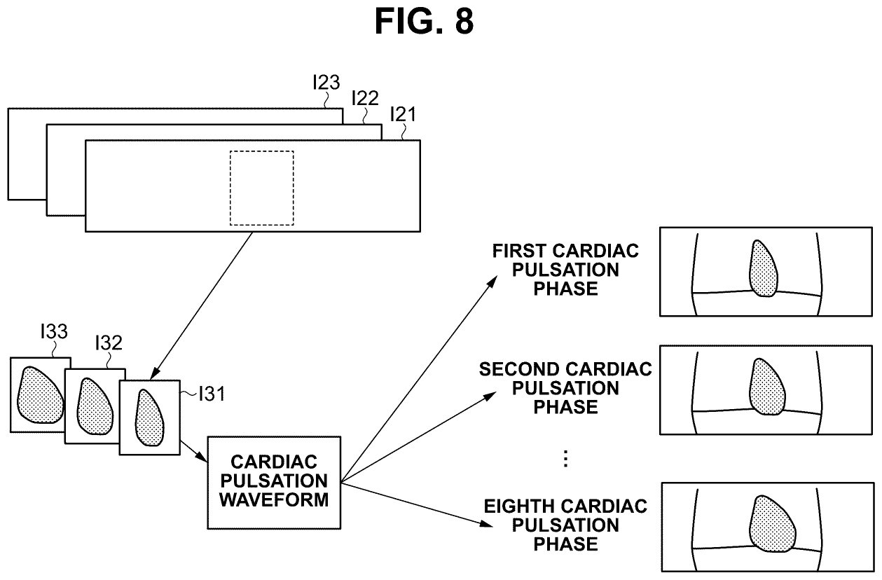

In estimating a cardiac pulsation waveform for each respiration phase, only the data near the heart may be processed. For example, as illustrated by the broken lines in FIG. 8, the second analysis function 125e subjects all of time-sharing reconstruction images including time-sharing reconstruction images I22 and I23 to processing for clipping the rectangular area including the heart in a time-sharing reconstruction image I21 as an image I31. The time-sharing reconstruction images I21, I22, and I23 are examples of reconstruction images based on partial data. Then, the second analysis function 125e estimates a cardiac pulsation waveform based on all of the clip images including the clip images I31, I32 and I33.

In step S106, the second image generation function 125f generates an image based on each of division data sets obtained by dividing the phase data for respective cardiac pulsation phases. For example, the second image generation function 125f divides the phase data for each cardiac pulsation phase based on the cardiac pulsation waveform estimated in step S105, and generates an image from each of the division data sets. The image generated in step S106 is an example of a second image.

A case where eight different phases are included in one period of the cardiac pulsation will be described below. Like the case of the respiration phase, this point can be suitably changed. More specifically, the number of phases included in one period of the cardiac pulsation and the definition of each phase can be suitably changed, automatically adjusted by the apparatus, or manually changed by the operator.

For example, the second image generation function 125f divides the second respiration phase data illustrated in FIG. 7 for each of the eight different cardiac pulsation phases, and generates an image for each cardiac pulsation phase. For example, as illustrated in FIG. 8, the second image generation function 125f generates eight images for eight cardiac pulsation phases: an image of the first cardiac pulsation phase, an image of the second cardiac pulsation phase, an image of the third cardiac pulsation phase, an image of the fourth cardiac pulsation phase, an image of the fifth cardiac pulsation phase, an image of the sixth cardiac pulsation phase, an image of the seventh cardiac pulsation phase, and an image of the eighth cardiac pulsation phase.

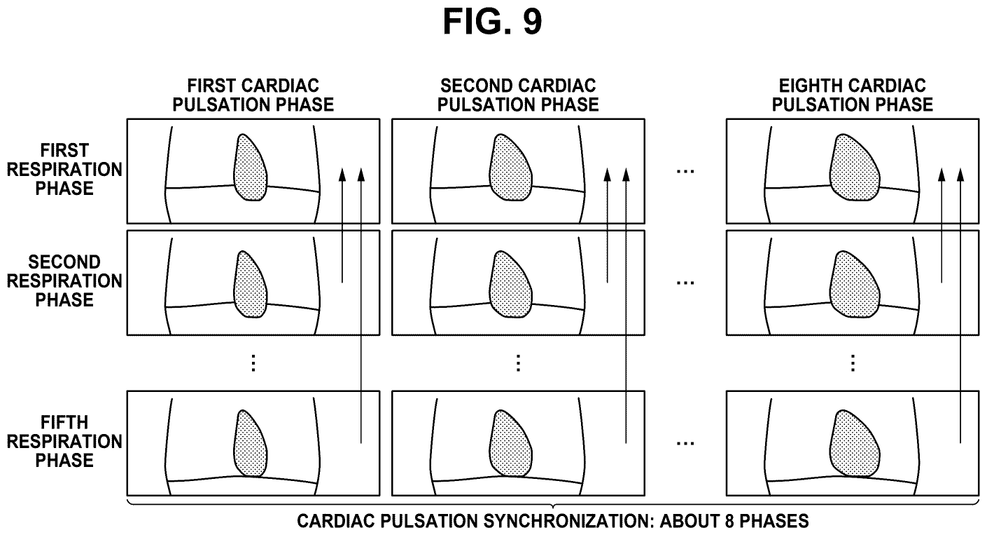

As described above, the second image generation function 125f generates eight images for respective cardiac pulsation phases based on the second respiration phase data. Likewise, the second image generation function 125f generates eight images for respective cardiac pulsation phases based on the first respiration phase data, eight images for respective cardiac pulsation phases based on third respiration phase data, eight images for respective cardiac pulsation phases based on fourth respiration phase data, and eight images for respective cardiac pulsation phases based on fifth respiration phase data. Thus, as illustrated in FIG. 9, the second image generation function 125f can generate a total of 40 images each of which has a different combination of the respiration phase and the cardiac pulsation phase.

Diagnoses such as heart function analysis may possibly be performed by using the images illustrated in FIG. 9 as they are. That is, according to the above processing of the first analysis function 125b, the first image generation function 125c, the second analysis function 125e and the second image generation function 125f, the synchronous reconstruction for periodic movements of a subject can be performed without using external devices. However, each image illustrated in FIG. 9 is generated based on a small number of time-division counts, and therefore affected by statistical noise. More specifically, each image illustrated in FIG. 9 including data having the number of counts that is about “1/40” of the total number of counts obtained through nuclear medicine scan, and therefore provides a relatively low image quality.

The movement of the subject P by the respiration is known to be small in the phase at the end of the exhalation. Therefore, if the first respiration phase is the phase at the end of the exhalation, for example, the eight images of the first respiration phase are relatively less defocused and are suitable for diagnoses. In other words, even if the 40 images illustrated in FIG. 9 are provided, 32 images other than the images of the first respiration phase may be unused for diagnoses. It is not preferable to perform useless calculation that generates images unused for diagnoses.

Providing the 40 images illustrated in FIG. 9 as they are is not preferable from the viewpoint of the image quality and calculation efficiency. In step S107, the totaling processing function 125g combines each group of images of different respiration phases and a common cardiac pulsation phase by applying a motion vector to each image, thus generating a single-phase image corresponding to a specific respiration phase.

For example, the totaling processing function 125g combines a group of five images of the first cardiac pulsation phase illustrated in FIG. 9 by applying a motion vector to each image. More specifically, the totaling processing function 125g applies a motion vector for performing mutual transformation between the first and the second respiration phases to images of the second respiration phase. The totaling processing function 125g also applies a motion vector for performing mutual transformation between the first and the third respiration phases to images of the third respiration phase. The totaling processing function 125g also applies a motion vector for performing mutual transformation between the first and the fourth respiration phases to images of the fourth respiration phase. The totaling processing function 125g also applies a motion vector for performing mutual transformation between the first and the fifth respiration phases to images of the fifth respiration phase. Further, the totaling processing function 125g combines the image of the first respiration phase and the image of the second respiration phase, the image of the third respiration phase, the image of the fourth respiration phase, and the image of the fifth respiration phase after motion vector application to generate a single-phase image corresponding to the first respiration phase.

As described above, the totaling processing function 125g combines a group of five images of the first cardiac pulsation phase to generate a single-phase image corresponding to the first respiration phase. Such a single-phase image is data for the number of counts corresponding to the five images of the first cardiac pulsation phase, and as a consequence, the influence of statistical noise is reduced to improve the image quality.

Likewise, the totaling processing function 125g combines a group of five images of the second cardiac pulsation phase to generate a single-phase image corresponding to the first respiration phase. Likewise, for each of the third to the eighth cardiac pulsation phases, the totaling processing function 125g generates a single-phase image corresponding to the first respiration phase.

In other words, for each of the eight different cardiac pulsation phases, the totaling processing function 125g can generate a single-phase image corresponding to the first respiration phase. These eight single-phase images are cardiac pulsation synchronous images which are usable for heart function analysis and other diagnoses. These eight single-phase images are generated by laconically using the 40 images illustrated in FIG. 9, each of which has a high image quality based on a sufficient number of counts. More specifically, the eight single-phase images generated by the totaling processing function 125g can improve the efficiency of heart function analysis and other diagnoses.

The single-phase images generated by the totaling processing function 125g are displayed, for example, on the display 123 under the control of the control function 125a. In addition, for example, the single-phase images generated by the totaling processing function 125g are transmitted to the PACS server under the control of the control function 125a and is kept.

Although FIG. 9 illustrates an example where single-phase images corresponding to the first respiration phase is generated, single-phase images corresponding to other respiration phases can also be generated. For example, the totaling processing function 125g combines each group of images of a common cardiac pulsation phase by applying motion vectors for performing mutual transformation between the second respiration phase and other phases. Thus, the totaling processing function 125g generates single-phase images corresponding to the second respiration phase. More specifically, the totaling processing function 125g can generate cardiac pulsation synchronous images for optional respiration phases.

The respiration phase to be subjected to the single-phase image generation may be preset or optionally selected by the operator. For example, the phase at the end of the exhalation in which the subject movement by the respiration is minimized may be selected as a respiration phase subjected to the single-phase image generation. For example, the respiration phase in which X-ray Computed Tomography (CT) images to be used for attenuation correction and scattered radiation correction of nuclear medicine images are captured may be selected as a respiration phase subjected to the single-phase image generation. A plurality of respiration phases to be subjected to the single-phase image generation may be selected.

The flowchart illustrated in FIG. 2 is to be considered as illustrative and may be suitably modified. For example, processing for acquiring the first motion vector in step S104 may be executed after heartbeat motion analysis in step S105 or second image generation in step S106, or executed in parallel with these steps.

As described above, the nuclear medicine diagnostic apparatus 10 according to the first exemplary embodiment includes the first analysis function 125b, the first image generation function 125c, the vector acquisition function 125d, the second analysis function 125e, the second image generation function 125f, and the totaling processing function 125g. The first analysis function 125b analyzes the respiration motion based on the list mode data obtained through nuclear medicine scan on the subject P having respiration motion and heartbeat motion. The first image generation function 125c generates a first image based on each of division data sets obtained by dividing the list mode data for respective respiration phases. The vector acquisition function 125d acquires a motion vector indicating variations between respiration phases based on the first image. The second analysis function 125e analyzes the heartbeat motion for each respiration phase based on the list mode data. The second image generation function 125f generates a second image based on each of division data sets obtained by dividing the phase data for respective cardiac pulsation phases, the phase data being obtained by dividing the list mode data for respective respiration phases. The totaling processing function 125g combines each group of second images of different respiration phases and a common cardiac pulsation phase by applying a motion vector to the second images. Thus, the totaling processing function 125g generates a single-phase image corresponding to a specific respiration phase. The above-described configuration allows the nuclear medicine diagnostic apparatus 10 to perform the synchronous reconstruction without using external devices for a subject with a plurality of movements such as the respiration motion and heartbeat motion.

The second analysis function 125e according to the first exemplary embodiment analyzes the heartbeat motion for each respiration phase based on the list mode data. For example, the second analysis function 125e extracts partial data from the phase data obtained by dividing the list mode data for respective respiration phases, and estimates a cardiac pulsation waveform based on a reconstruction image reconstructed based on the extracted partial data. This enables the second analysis function 125e to eliminate or prevent the influence of the respiration motion in heartbeat motion analysis and accurately estimate a cardiac pulsation waveform.

As described above, the movement of the respiration motion is larger than the heartbeat motion. If the list mode data is analyzed as it is, the respiration motion is predominantly detected, making it difficult to estimate a cardiac pulsation waveform without using external devices. On the other hand, the nuclear medicine diagnostic apparatus 10 according to the first exemplary embodiment performs heartbeat motion analysis on the phase data obtained by dividing the list mode data for respective respiration phases. This enables accurately estimating a cardiac pulsation waveform without using external devices, thus implementing the data-driven cardiac pulsation synchronous reconstruction.

The second analysis function 125e according to the first exemplary embodiment extracts a part of the phase data obtained by dividing the list mode data for respective respiration phases, as partial data. Then, the second analysis function 125e clips the region corresponding to the heart out of the reconstruction image reconstructed based on the extracted partial data as a clip image, and estimates a cardiac pulsation waveform based on the clip image. The above-described configuration enables extracting only data near the heart and analyzing the cardiac pulsation, making it possible to restrict the calculation cost and improve the analysis accuracy.

### Second Exemplary Embodiment

The first exemplary embodiment has been described above centering on an example of generating a single-phase image corresponding to a specified phase of the respiration phases, as the single-phase image illustrated in step S107 in FIG. 2. In contrast, a second exemplary embodiment will be described below centering on an example variation of the single-phase image generated in step S107.

The nuclear medicine diagnostic apparatus 10 according to the second exemplary embodiment can be configured similarly to the nuclear medicine diagnostic apparatus 10 illustrated in FIG. 1, and is different in part of the processing performed by the processing circuitry 125 from the first exemplary embodiment. The following describes differences in the processing performed by the processing circuitry 125 from the first exemplary embodiment, and duplicated descriptions will be omitted. Unless otherwise noted, the descriptions according to the first exemplary embodiment are also applied to the second exemplary embodiment.

The processing circuitry 125 according to the second exemplary embodiment executes processing similar to steps S101, S102, S103, S105, and S106 illustrated in FIG. 2 to generate images for respective respiration phases and respective cardiac pulsation phases. For example, like the case illustrated in FIG. 9, the processing circuitry 125 generates 40 images corresponding to combinations of eight cardiac pulsation phases and five respiration phases.

According to the second exemplary embodiment, step S104 illustrated in FIG. 2 may be omitted. More specifically, the vector acquisition function 125d according to the second exemplary embodiment does not need to acquire the first motion vector indicating variations between respiration phases.

The vector acquisition function 125d according to the second exemplary embodiment acquires the second motion vector indicating variations between cardiac pulsation phases based on the second image. For example, the vector acquisition function 125d performs registration processing between the second images of a common respiration phase and different cardiac pulsation phases, and acquires the inverse transformation of the registration processing as a second motion vector. The vector acquisition function 125d may perform the registration processing or second motion vector acquisition processing itself through a machine learning technique (such as neural networks).

For example, the totaling processing function 125g according to the second exemplary embodiment generates a single-phase image corresponding to a specific cardiac pulsation phase for respective respiration phases as the single-phase image in step S107. More specifically, the totaling processing function 125g combines each group of images of different cardiac pulsation phases and a common respiration phase by applying the second motion vector indicating variations between cardiac pulsation phases. Thus, the totaling processing function 125g generates a single-phase image corresponding to a specific cardiac pulsation phase.

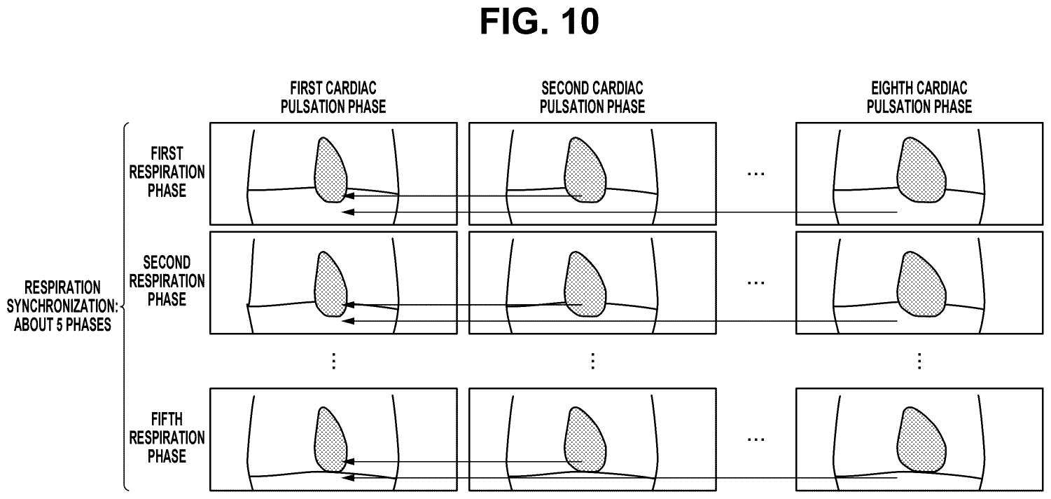

For example, as illustrated in FIG. 10, the totaling processing function 125g combines a group of eight images of the first respiration phase to generate a single-phase image corresponding to the first cardiac pulsation phase. Likewise, for each of the second to the fifth respiration phases, the totaling processing function 125g generates a single-phase image corresponding to the first cardiac pulsation phase. More specifically, the totaling processing function 125g can generate a single-phase image corresponding to the first cardiac pulsation phase for each of the five respiration phases. These five single-phase images are respiration synchronous images which are usable for lung function analysis and other diagnoses.

For another example, the totaling processing function 125g according to the second exemplary embodiment generates a single image corresponding to a specific respiration phase and a specific cardiac pulsation phase as the single-phase image in step S107.

More specifically, like the case illustrated in FIG. 9, the totaling processing function 125g combines each group of images of different respiration phases and a common cardiac pulsation phase by applying the first motion vector for performing mutual transformation between the first respiration phase and other phases. Thus, the totaling processing function 125g generates eight single-phase images corresponding to the first respiration phase. Further, the totaling processing function 125g combines the eight single-phase images corresponding to the first respiration phase by applying the second motion vector for performing mutual transformation between the first cardiac pulsation phase and other phases. Thus, the totaling processing function 125g generates a single image corresponding to the first respiration phase and the first cardiac pulsation phase.

In such a case, the vector acquisition function 125d may acquire the second motion vector directly from the second images for respective respiration phases and respective cardiac pulsation phases, and acquire the second motion vector based on a single-phase image corresponding to a specific respiration phase.

For example, the processing in step S106 in FIG. 2 generates the second images for respective respiration phases and respective cardiac pulsation phases. Then, the vector acquisition function 125d acquires the second motion vector indicating variations between cardiac pulsation phases based on the second image. The totaling processing function 125g also combines each group of second images of different respiration phases and a common cardiac pulsation phase by applying the first motion vector. Thus, the totaling processing function 125g generates eight single-phase images corresponding to the first respiration phase. Further, the totaling processing function 125g combines the eight single-phase images corresponding to the first respiration phase by applying the second motion vector. Thus, the totaling processing function 125g generates a single image corresponding to the first respiration phase and the first cardiac pulsation phase.

For another example, after the second images for respective respiration phases and respective cardiac pulsation phases are generated, the totaling processing function 125g combines each group of second images of different respiration phase and a common cardiac pulsation phase by applying the first motion vector. Thus, the totaling processing function 125g generates eight single-phase images corresponding to the first respiration phase. Then, the vector acquisition function 125d acquires the second motion vector indicating variations between cardiac pulsation phases based on the eight single-phase images. Further, the totaling processing function 125g combines eight single-phase images corresponding to the first respiration phase by applying the second motion vector. Thus, the totaling processing function 125g generates a single image corresponding to the first respiration phase and the first cardiac pulsation phase.

Alternatively, like the case illustrated in FIG. 10, the processing circuitry 125 combines each group of images of different cardiac pulsation phases and a common respiration phase by applying the second motion vector for performing mutual transformation between the first cardiac pulsation phase and other phases. Thus, the processing circuitry 125 generates five single-phase images corresponding to the first cardiac pulsation phase. Further, the totaling processing function 125g combines five single-phase images corresponding to the first cardiac pulsation phase by applying the first motion vector for performing mutual transformation between the first respiration phase and other phases. Thus, the totaling processing function 125g generates a single image corresponding to the first respiration phase and the first cardiac pulsation phase.

Such a single image has a high image quality based on a larger number of counts. The above-described example generates a single image corresponding to the first respiration phase and the first cardiac pulsation phase. However, single images corresponding to other respiration phases and cardiac pulsation phases can also be generated. For example, when a single image is generated for each cardiac pulsation phase, the plurality of single images are cardiac pulsation synchronous images. When a single image is generated for each respiration phase, the plurality of single images are respiration synchronous images.

### Third Exemplary Embodiment

The present disclosure may be implemented in various different forms in addition to the above-described first and second exemplary embodiments.

For example, FIG. 1 illustrates the nuclear medicine diagnostic apparatus 10 as a PET apparatus. However, the exemplary embodiments are not limited thereto. The above-mentioned exemplary embodiments are similarly applicable even in a case where the nuclear medicine diagnostic apparatus 10 is an SPECT apparatus.

According to the above-described exemplary embodiments, the control function 125a, the first analysis function 125b, the first image generation function 125c, the vector acquisition function 125d, the second analysis function 125e, the second image generation function 125f, and the totaling processing function 125g are executed by the processing circuitry 125 included in the nuclear medicine diagnostic apparatus 10. However, the exemplary embodiments are not limited thereto. A part or all of the functions of the processing circuitry 125 may be executed by processing circuitry included in apparatuses other than the nuclear medicine diagnostic apparatus 10.

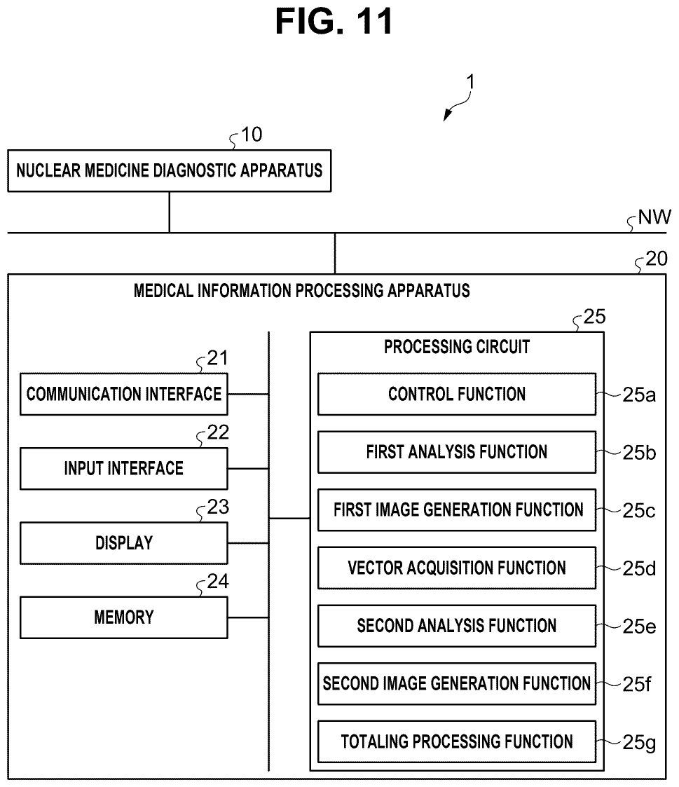

FIG. 11 illustrates a medical information processing system 1 including the nuclear medicine diagnostic apparatus 10 and a medical information processing apparatus 20. In the medical information processing system 1, for example, the nuclear medicine diagnostic apparatus 10 and the medical information processing apparatus 20 are connected via the network NW, and the processing circuitry 25 included in the medical information processing apparatus 20 may perform similar processing to the processing circuitry 125.

The medical information processing apparatus 20 illustrated in FIG. 11 includes a communication interface 21, an input interface 22, a display 23, a memory 24, and processing circuitry 25. The communication interface 21, the input interface 22, the display 23, and the memory 24 can be similarly configured to the communication interface 121, the input interface 122, the display 123, and the memory 124, respectively, illustrated in FIG. 1, and redundant descriptions thereof will be omitted. The processing circuitry 25 includes a control function 25a, a first analysis function 25b, a first image generation function 25c, a vector acquisition function 25d, a second analysis function 25e, a second image generation function 25f, and a totaling processing function 25g.

For example, the control function 25a controls transmission and reception of data via the network NW. For example, the control function 25a acquires the list mode data collected by the nuclear medicine diagnostic apparatus 10, via the network NW. For example, the control function 25a transmits the list mode data and nuclear medicine images reconstructed based on the list mode data, to the PACS server for registration. The control function 25a also performs controls display of the display 123. The first analysis function 25b, the first image generation function 25c, the vector acquisition function 25d, the second analysis function 25e, the second image generation function 25f, and the totaling processing function 25g have similar functions to the first analysis function 125b, the first image generation function 125c, the vector acquisition function 125d, the second analysis function 125e, the second image generation function 125f, and the totaling processing function 125g, respectively, illustrated in FIG. 1.

The word “processor” used in the above descriptions means such circuitry as a CPU, GPU, ASIC, Programmable Logic Device (PLD) (including a Simple Programmable Logic Device (SPLD), Complex Programmable Logic Device (CPLD), and Field Programmable Gate Array (FPGA)). For example, if a processor is a CPU, the processor implements its function by reading a program stored in storage circuitry and executing the program. If a processor is an ASIC, the program is not stored in the storage circuitry. Instead, the function is directly built in logic circuitry in the circuitry of the processor. Each processor according to the exemplary embodiments is not necessarily configured as single circuitry. A plurality of independent circuits may be combined to form one processor and implement its function. Further, a plurality of components in each drawing may be integrated into one processor and implement its function.

Components of each apparatus according to the above-described exemplary embodiments are to be functionally conceptual, and does not necessarily need to be physically configured as illustrated in the drawings. More specifically, specific forms of distribution and integration of different apparatuses are not limited to the illustrations. All or a part of the apparatuses can be configured in functionally or physically distributed and integrated forms in desired units depending on various loads and usage situations. Further, all or any part of processing functions of each apparatus may be implemented by a CPU and programs analyzed and executed by the CPU, or implemented as wired logic hardware.

Methods according to the above-described exemplary embodiments can be implemented when a computer such as a personal computer and a workstation executes a prepared program. This program can be distributed via a network such as the Internet. This medical information processing program is recorded in a computer-readable non-transitory recording medium such as a hard disk, flexible disk (FD), compact disc read only memory (CD-ROM), magneto-optical disk (MO), and digital versatile disc (DVD), and can be read from the recording medium and executed by a computer.

According to at least one of the above-described exemplary embodiments, the synchronous reconstruction for periodic movements of a subject can be performed without using external devices.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A nuclear medicine diagnostic apparatus comprising processing circuitry configured to:
analyze respiration motion based on list mode data obtained through nuclear medicine scan on a subject having respiration motion and heartbeat motion;
generate first images from respective division data sets obtained by dividing the list mode data for respective respiration phases as phases of the respiration motion;
analyze the heartbeat motion for the respective respiration phases based on the list mode data; and
generate second images from respective division data sets obtained by dividing phase data, obtained by dividing the list mode data for the respective respiration phases, for respective cardiac pulsation phases as phases of the heartbeat motion.

2. The nuclear medicine diagnostic apparatus according to claim 1, wherein the processing circuitry is further configured to:
acquire at least either one of a first motion vector indicating variations between the respiration phases and a second motion vector indicating variations between the cardiac pulsation phases; and
generate first single-phase images corresponding to a specific respiration phase by applying the first motion vector to the second images in each group of the second images of different respiration phases and a common cardiac pulsation phase and combining them, or generate second single-phase images corresponding to a specific cardiac pulsation phase by applying the second motion vector to the second images in each group of the second images of different cardiac pulsation phases and a common respiration phase and combining them.

3. The nuclear medicine diagnostic apparatus according to claim 2, wherein the processing circuitry is further configured to:
generate the first single-phase images by applying the first motion vector to the second images in each group of the second images of different respiration phases and a common cardiac pulsation phase and combining them;
generate the second single-phase images by applying the second motion vector to the first single-phase images and combining them, or by applying the second motion vector to the second images in each group of the second images of different cardiac pulsation phases and a common respiration phase and combining them; and
generate a single image corresponding to a specific respiration phase and a specific cardiac pulsation phase by applying the first motion vector to the second single-phase images and combining them.

4. The nuclear medicine diagnostic apparatus according to claim 1, wherein the processing circuitry is further configured to:
extract a part of the list mode data in the time direction as partial data, and estimate a respiration waveform as a waveform of the respiration motion based on a reconstruction image reconstructed based on the extracted partial data; and
generate the first images from each of division data sets obtained by dividing the list mode data for respective respiration phases based on the respiration waveform.

5. The nuclear medicine diagnostic apparatus according to claim 4, wherein the processing circuitry is further configured to extract the partial data having a time duration with a first length from the list mode data at time intervals with a second length shorter than the first length.

6. The nuclear medicine diagnostic apparatus according to claim 1, wherein the processing circuitry is further configured to:
extract a part of the phase data in the time direction as partial data, and estimate a cardiac pulsation waveform as a waveform of the heartbeat motion based on a reconstruction image reconstructed based on the extracted partial data; and
generate the second images from each of division data sets obtained by dividing the phase data for respective cardiac pulsation phases based on the cardiac pulsation waveform.

7. The nuclear medicine diagnostic apparatus according to claim 2, wherein the processing circuitry is further configured to:
acquire the first motion vector based on the first images, and
generate first single-phase images corresponding to a specific respiration phase by applying the first motion vector to the second images in each group of the second images of different respiration phases and a common cardiac pulsation phase and combining them.

8. The nuclear medicine diagnostic apparatus according to claim 2,
acquire the second motion vector based on the second images, and
generate second single-phase images corresponding to a specific cardiac pulsation phase by applying the second motion vector to the second images in each group of the second images of different cardiac pulsation phases and a common respiration phase and combining them.
